(19) United States
(12) Plant Patent Application Publication
(10) Pub. No.: US 2016/0309632 P1
(43) Pub. Date: Oct. 20, 2016

(54) Female pistachio variety named 'GUMDROP'

(50) ;

(71) Applicant: The Regents of the University of California, Oakland, CA (US)

(72) Inventors: Craig E. Kallsen, Davis, CA (US); Dan E. Parfitt, Davis, CA (US)

(21) Appl. No.: 14/999,158

(22) Filed: Apr. 4, 2016

Related U.S. Application Data
(60) Provisional application No. 62/147,539, filed on Apr. 14, 2015.

(51) Int. Cl. A01H 5/00

(52) U.S. Cl. PLT/152

(57) ABSTRACT

A new female pistachio variety (Pistacia vera L.) designated as ‘Gumdrop’ is provided. This variety exhibits earlier flowering and earlier harvest dates compared to the ‘Golden Hills’ and industry standard ‘Kerman’ cultivars.

### CROSS-REFERENCE TO RELATED APPLICATION

This application claims the benefit of U.S. Provisional Application No. 62/147,539, filed Apr. 14, 2015, which is incorporated herein by reference in its entirety.

### LATIN NAME

Botanical/commercial classification: (Pistacia vera L.), new pistachio variety.

### VARIETAL DENOMINATION

The varietal denomination of the claimed pistachio variety is ‘Gumdrop’.

### BACKGROUND OF THE INVENTION

An objective of pistachio breeding programs is to develop new varieties that can be harvested at unique times relative to other pistachio varieties. The female pistachio variety ‘Kerman’ is the main later-season pistachio cultivar grown in California and in other parts of the world, but other female pistachio varieties are also grown, such as ‘Golden Hills’. A major problem for pistachio growers that has developed with the rapid increase in pistachio plantings in California, for example, is the availability of harvest equipment/contractors and processing capacity, since most of the existing crop (‘Kerman’) matures at about the same time. The pistachio industry has ˜40% of its orchards in non-bearing status, yet plantings have not slowed. Difficulty finding both harvesting equipment and people to run them at harvest time is a problem for pistachio growers. The industry-wide harvest window is short because of the large amount of future bearing ‘Kerman’ plantings, all of which matures at about the same time. Nut processing facilities will likely need to greatly increase their capacity to handle this large increase in nuts, yet will use this increased capacity for a very short time period.

Further, ‘Kerman’ appears to be vulnerable to lack of winter chill, as shown directly through erratic bloom and indirectly from lack of overlap with this variety's pollenizer, ‘Peters’. Low chill years have presented great difficulties for pistachio growers in the lower San Joaquin valley of California during periods when chilling was well below the minimum needed for synchronous flower development in ‘Kerman’. High levels of “blanking” and non-splits were observed.

‘Golden Hills’ has become a popular alternative to ‘Kerman’ because it can be harvested before ‘Kerman’. However, it would be advantageous to have additional varieties that can be harvested even earlier than ‘Golden Hills’ to increase the availability of pistachio harvesting equipment and processing capacity, as well as to have different pistachio varieties available that can be harvested in a maturity series. Developing a harvest date series is an optimal way to use existing harvest equipment/contractor and processing plant resources. Without staggered harvest dates, the pistachio industry will have to develop significantly increased (e.g. 2×) harvesting capability (which is now provided by custom harvestors) and find investors willing to fund new processing plants (which are only used for a few weeks per year). Failure to develop these facilities or alternatively, a harvest date series, will expose the industry to significant risk from aflatoxin contamination in the crop due to extended pre-processing times.

Thus, there exists a need for improved pistachio varieties with earlier harvest dates than the present industry standards. The present female pistachio variety ‘Gumdrop’ described herein is a product of the breeding efforts to produce improved pistachio varieties.

### SUMMARY OF THE INVENTION

The present invention relates to a new and distinct pistachio cultivar (Pistacia vera L.) which has been denominated as ‘Gumdrop’, and more particularly as a female pistachio variety which exhibits earlier flowering and earlier harvest dates compared to the ‘Golden Hills’ and industry standard ‘Kerman’ cultivars.

‘Gumdrop’ produces a similar yield and percentage of split, edible nuts as ‘Golden Hills’ while maintaining a similar low percentage of loose shells and kernels. Nut quality and processed nut appearance of ‘Gumdrop’ is similar to other cultivars. Further, ‘Gumdrop’ exhibits very good yield, commercial level nut characteristics, and early harvest. ‘Gumdrop’ blooms about five days before ‘Golden Hills’ and 10-11 days before ‘Kerman’. ‘Gumdrop’ matures about 12 days before ‘Golden Hills’ and about 24 days before ‘Kerman’. The harvest date for ‘Gumdrop’ is about 10-12 days earlier than ‘Golden Hills’. ‘Gumdrop’, ‘Golden Hills’, and ‘Kerman’ form a maturity series that spans nearly a month in the fall harvest season.

The earlier harvest date of ‘Gumdrop’ will permit pistachio growers to extend their harvest period and reduce competition for scarce harvesting resources. The earlier maturing date of ‘Gumdrop’ also makes it less susceptible to low chill effects and insect damage. Indeed, experience with ‘Golden Hills’ has shown that earlier harvest limits exposure of the crop to the last Navel Orangeworm flight, thereby significantly limiting potential aflatoxin contamination and crop loss issues. ‘Gumdrop’ has the earliest harvestable crop of any commercial cultivars, and also one of the earliest flowering times, suggesting that it may need less chilling than other commercial cultivars. This may be a very important characteristic in the future with respect to the warming climate. The very early harvest date of ‘Gumdrop’ also allows for the potential of high yield in future years, as well as the potential for significant yield a year earlier after planting than other planted cultivars. This would provide growers with more rapid return on equity.

‘Gumdrop’ was originally isolated as an open-pollinated offspring of ‘B15-69’, the open-pollination having taken place in a pistachio breeding program test plot near Famoso, Calif. during Year 0. The initial seedling was originally designated as ‘S-43’. Following initial identification, selection ‘S-43’ was later grown in test plots, and has been asexually propagated in California, USA. The first asexual propagation of ‘S-43’ took place in August of Year 7. Buds of ‘S-43’ were removed and grafted (using T-buds) into UCB-1 seedling rootstocks at a trial plot located in Buttonwillow, Calif. Replicated test plots for growing selection ‘S-43’ were established in Year 7 and Year 10, with evaluation data being collected during Year 12-Year 14. Selection ‘S-43’ was chosen as a candidate for release under the variety name ‘Gumdrop’.

### BRIEF DESCRIPTION OF THE DRAWINGS

FIG. 1A illustrates mean days to harvest for ‘Kerman’, ‘Golden Hills’, and ‘Gumdrop’ over several years (Years 12-14). FIG. 1B illustrates mean days to harvest for ‘Kerman’, ‘Golden Hills’, and ‘Gumdrop’ (‘S-43’) over Years 12-15. Horizontal bar for each grouping of varieties denotes the grand harvest date mean for that variety over Years 12-15. FIG. 1C illustrates combined harvest date data for all years (Years 12-15) by cultivar fitted to normal distributions.

FIG. 2A illustrates payable yield (lb/ac) for ‘Golden Hills’, ‘Kerman’, and ‘Gumdrop’ over several years (Years 12-14). FIG. 2B illustrates payable yield (lb/ac) for ‘Golden Hills’, ‘Kerman’, ‘Gumdrop’ (‘S-43’), and ‘S-32’ over several years (Years 12-15). FIG. 2C illustrates mean payable yield (lb/ac) for ‘Golden Hills’, ‘Kerman’, ‘Gumdrop’ (‘S-43’), and ‘S-32’ over several years (Years 12-15).

FIG. 3A illustrates fraction of split nuts for ‘Golden Hills’, ‘Kerman’, and ‘Gumdrop’ over several years (Years 12-14). FIG. 3B illustrates fraction of split nuts for ‘Golden Hills’, ‘Kerman’, ‘Gumdrop’ (‘S-43’), and ‘S-32’ over several years (Years 12-15). FIG. 3C illustrates an analysis of means for split nut fraction for ‘Golden Hills’, ‘Kerman’, ‘Gumdrop’ (‘S-43’), and ‘S-32’ over several years (Years 12-15).

FIG. 4A illustrates fraction of blank nuts for ‘Golden Hills’, ‘Kerman’, and ‘Gumdrop’ over several years (Years 12-14). FIG. 4B illustrates fraction of blank nuts for ‘Golden Hills’, ‘Kerman’, ‘Gumdrop’ (‘S-43’), and ‘S-32’ over several years (Years 12-15). FIG. 4C illustrates an analysis of means for blank nut fraction for ‘Golden Hills’, ‘Kerman’, ‘Gumdrop’ (‘S-43’), and ‘S-32’ over several years (Years 12-15).

FIG. 5A illustrates bug damage for ‘Golden Hills’, ‘Kerman’, and ‘Gumdrop’ over several years (Years 12-14). FIG. 5B illustrates insect damage fraction for ‘Golden Hills’, ‘Kerman’, ‘Gumdrop’ (‘S-43’), and ‘S-32’ over several years (Years 12-15). FIG. 5C illustrates an analysis of means for insect damage for ‘Golden Hills’, ‘Kerman’, ‘Gumdrop’ (‘S-43’), and ‘S-32’ over several years (Years 12-15).

FIG. 6A illustrates stain fraction for ‘Golden Hills’, ‘Kerman’, and ‘Gumdrop’ over several years (Years 12-14). FIG. 6B illustrates stain fraction for ‘Golden Hills’, ‘Kerman’, ‘Gumdrop’ (‘S-43’), and ‘S-32’ over several years (Years 12-15). FIG. 6C illustrates an analysis of means for stain fraction for ‘Golden Hills’, ‘Kerman’, ‘Gumdrop’ (‘S-43’), and ‘S-32’ over several years (Years 12-15).

FIG. 7A illustrates loose shells for ‘Golden Hills’, ‘Kerman’, and ‘Gumdrop’ over several years (Years 12-14). FIG. 7B illustrates loose shells for ‘Golden Hills’, ‘Kerman’, ‘Gumdrop’ (‘S-43’), and ‘S-32’ over several years (Years 12-15). FIG. 7C illustrates an analysis of means for loose shells for ‘Golden Hills’, ‘Kerman’, ‘Gumdrop’ (‘S-43’), and ‘S-32’ over several years (Years 12-15).

FIG. 8A illustrates nut weight for ‘Golden Hills’, ‘Kerman’, and ‘Gumdrop’ over several years (Years 12-14). FIG. 8B illustrates nut weight for ‘Golden Hills’, ‘Kerman’, ‘Gumdrop’ (‘S-43’), and ‘S-32’ over several years (Years 12-15). FIG. 8C illustrates an analysis of means for nut weight for ‘Golden Hills’, ‘Kerman’, ‘Gumdrop’ (‘S-43’), and ‘S-32’ over several years (Years 12-15).

FIG. 9A illustrates the trunk, branches, and canopy of ‘Gumdrop’. FIG. 9B illustrates the leaves of ‘Gumdrop’.

FIG. 10 illustrates the relative state of ‘Gumdrop’ flower development vs. ‘Golden Hills’ and ‘Kerman’.

FIG. 11A illustrates ‘Gumdrop’ nut clusters at maturity, showing the general size and shape of the clusters (e.g. number of nuts and distribution in the clusters). FIG. 11B illustrates an additional view of ‘Gumdrop’ nut clusters.

FIG. 12A, FIG. 12B, and FIG. 12C illustrate the appearance of husked and dried nuts of ‘Gumdrop’ (FIG. 12A), ‘Kerman’ (FIG. 12B), and ‘Golden Hills’ (FIG. 12C).

FIG. 13 illustrates the relative size and appearance of isolated kernels of ‘Gumdrop’ and ‘Kerman’.

FIG. 14A illustrates nut length (mm) for ‘Golden Hills’, ‘Kerman’, ‘Gumdrop’ (‘S-43’), and ‘S-32’ over several years (Years 13-15). FIG. 14B illustrates an analysis of means for nut length (mm) for ‘Golden Hills’, ‘Kerman’, ‘Gumdrop’ (‘S-43’), and ‘S-32’ over several years (Years 13-15).

FIG. 15A illustrates nut width (mm) for ‘Golden Hills’, ‘Kerman’, ‘Gumdrop’ (‘S-43’), and ‘S-32’ over several years (Years 13-15). FIG. 15B illustrates an analysis of means for nut width (mm) for ‘Golden Hills’, ‘Kerman’, ‘Gumdrop’ (‘S-43’), and ‘S-32’ over several years (Years 13-15).

### DETAILED BOTANICAL DESCRIPTION

The following is a detailed botanical description of the new female pistachio cultivar designated as ‘Gumdrop’, including the key differentiating characteristics of this variety and comparisons of certain characteristics of ‘Gumdrop’ to other pistachio varieties.

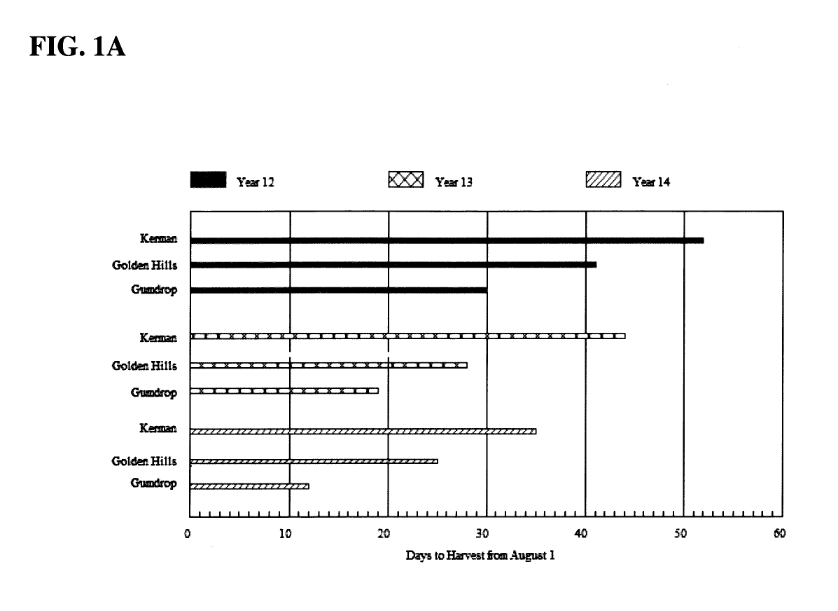

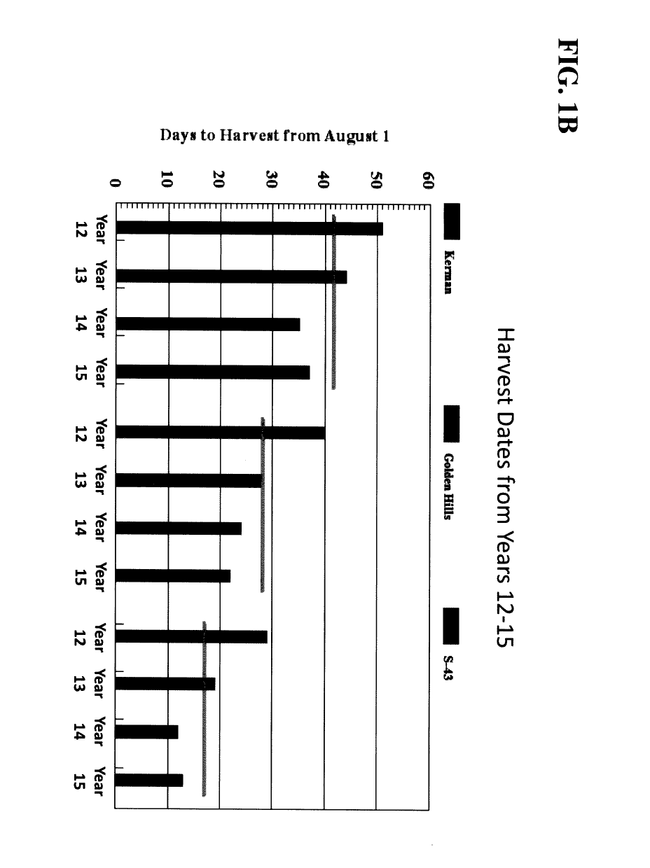

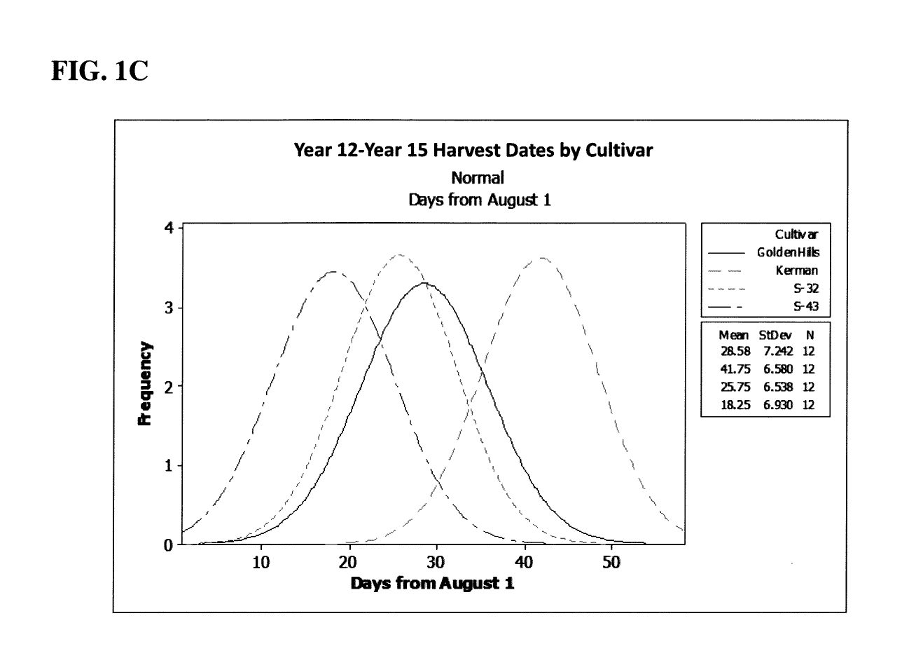

Harvest Date: ‘Gumdrop’ matures much earlier than ‘Golden Hills’ and ‘Kerman’. The harvest date for ‘Gumdrop’ is about 10-12 days earlier than ‘Golden Hills’ and 24 days before ‘Kerman’ (FIG. 1A). This is a valuable commercial characteristic, as it allows increased availability of harvest equipment/contractors and processing capacity. FIG. 1B illustrates harvest date data for multiple female selections, including ‘Gumdrop’ (‘S-43’), during Years 12-15. FIG. 1C demonstrates how ‘Gumdrop’ fits into a harvest series for the pistachio industry, and further illustrates the earlier average harvest time of this variety as compared to either ‘Kerman’ or ‘Golden Hills’.

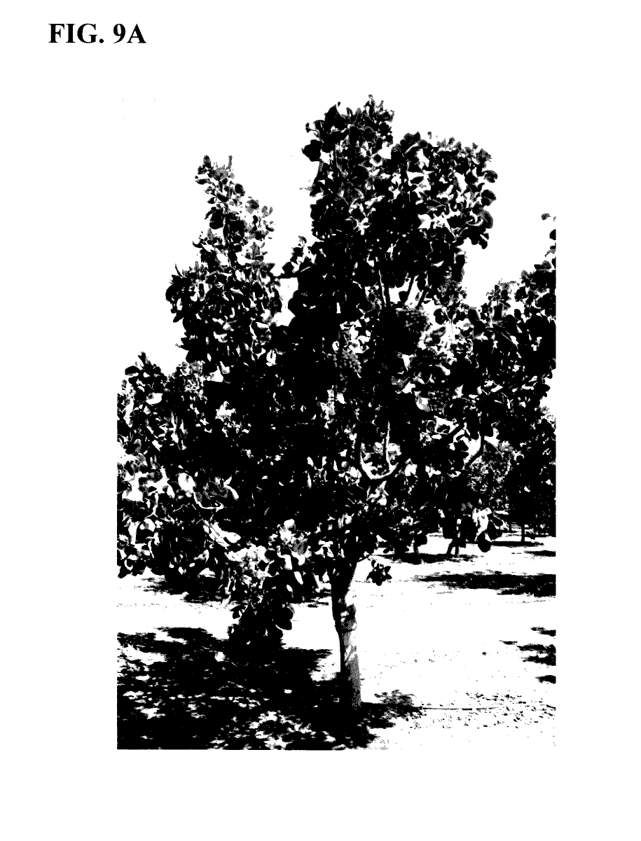

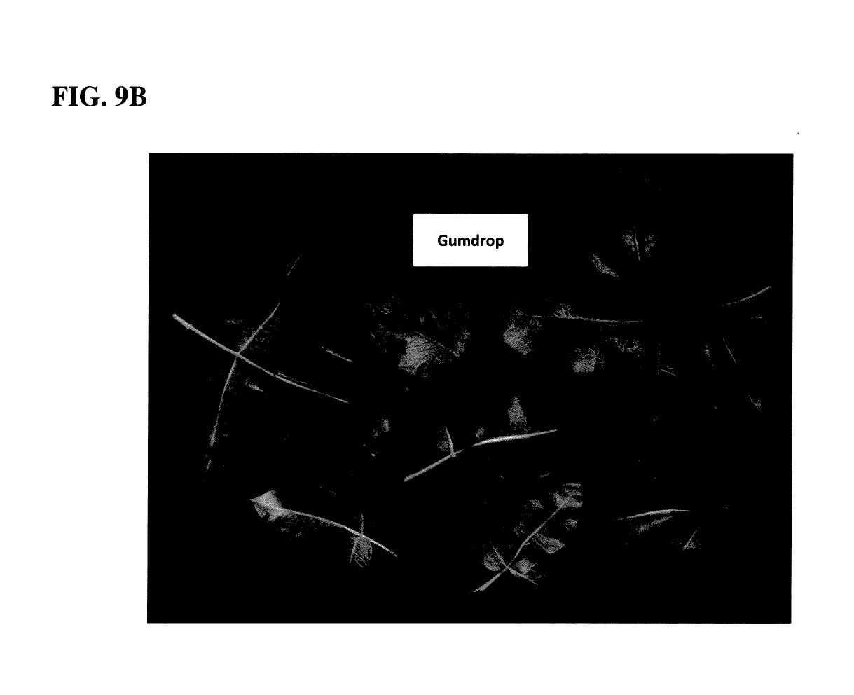

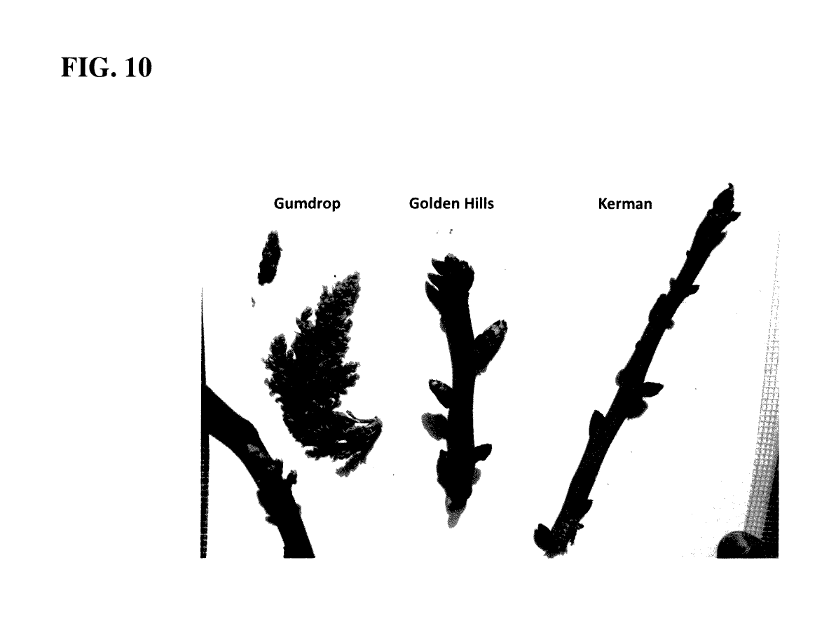

Plant and Flowering Time: ‘Gumdrop’ is at full bloom five days before ‘Golden Hills’ and 10-11 days before ‘Kerman’. An image of a ‘Gumdrop’ tree, including images of the trunk, branches, and canopy, is presented in FIG. 9A. An image of the leaves of ‘Gumdrop’ is presented in FIG. 9B. The earlier development of flowers in ‘Gumdrop’ as compared to ‘Golden Hill's and ‘Kerman’ is also presented in FIG. 10.

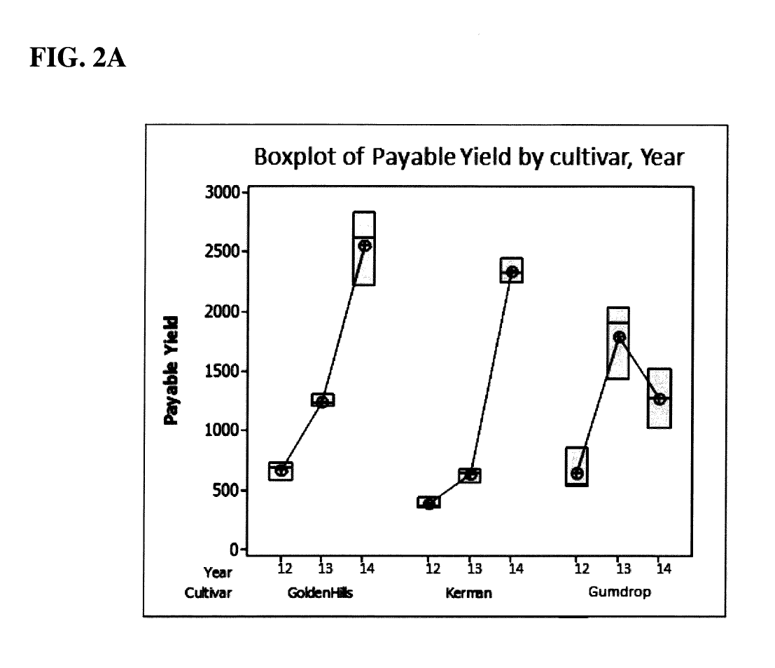

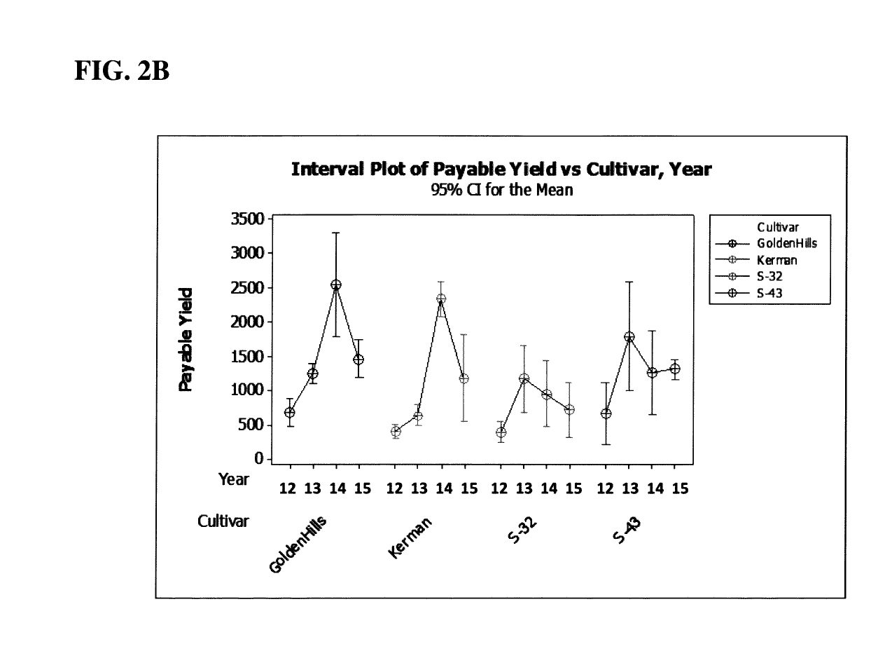

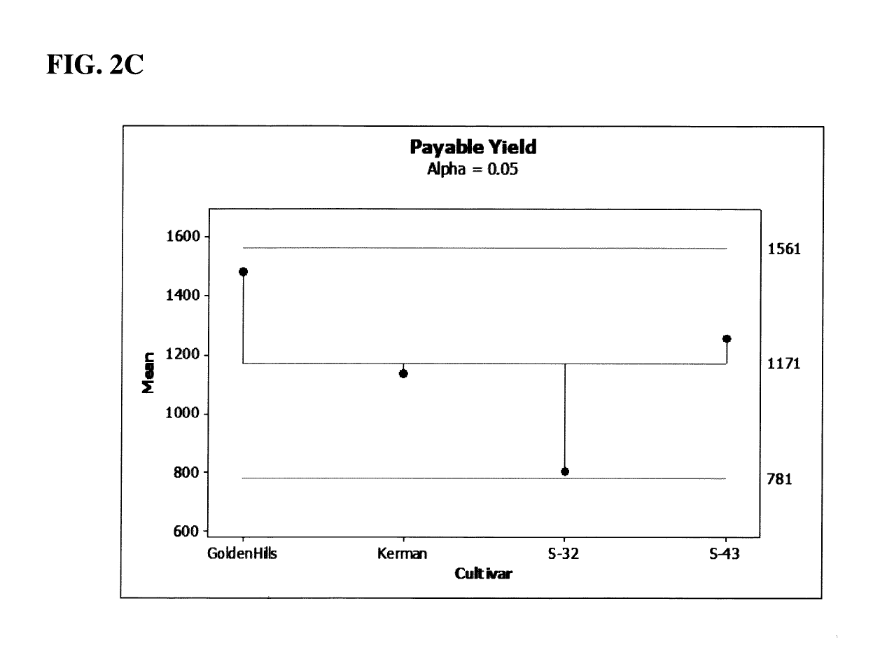

Yield: Total edible yield is the weight of in-shell edible split nuts (nuts and shell) in addition to the kernels that come from shelling stock and closed shells. This is also called grower paid yield or payable yield. ‘Gumdrop’ yielded significantly more nuts than ‘Kerman’ (2.4×) and ‘Golden Hills’ (1.3×) in the first two years of replicated trials. ‘Gumdrop’ had a mean payable yield of 1239 lb/ac over the first 3 years of harvested yield (Years 12-14), similar to ‘Golden Hills’ (1492 lb/ac) and better but not significantly different (Bonferroni, Tukey tests) yield than ‘Kerman’ (1121 lb/ac) (FIG. 2A). ‘Gumdrop’ yield was reduced in Year 14 due to high yield in Year 13, while ‘Golden Hills’ and ‘Kerman’ did not begin to bear significantly until the third harvest (Year 14) (8^{th} leaf) (FIG. 2A). These differences reflect better early yield for ‘Gumdrop’ and consequent earlier initiation of the alternate bearing cycle.

Based on data from Years 12-15, ‘Gumdrop’ had a mean payable yield of 1256 lb/ac over the first 4 years of harvested yield (Years 12-15), less than ‘Golden Hills’ (1484 lb/ac) and better but not significantly different (Bonferroni, Tukey) than ‘Kerman’ (1134 lb/ac). An analysis and statistics summary is provided in Table 1A and Table 1B. The 95% confidence interval plot in FIG. 2B shows that ‘Gumdrop’ (‘S-43’) yield was reduced in Year 14 due to high yield in Year 13, but had better yield in Year 15 than ‘Kerman’, suggesting that it may be less susceptible to alternate bearing than ‘Kerman’ or ‘Golden Hills’, with performance characteristics more typical of ‘Lost Hills’. All values are based on an industry harvest practice of shaking the trees to obtain the nuts. No attempt was made to remove any adhering nuts after shaking. Mean payable yields for the various varieties analyzed during Years 12-15 are shown in FIG. 2C.

TABLE 1A
Analysis of Variance for Payable Yield

| Source | DF | MS | F | P |
|---|---|---|---|---|
| Cultivar | 3 | 954866 | 28.50 | 0.000 |
| Year | 3 | 3149551 | 94.02 | 0.000 |
| Cultivar * Year | 9 | 654280 | 19.53 | 0.000 |
| Block | 2 | 4915 | 0.15 | 0.864 |
| Error | 30 | 33500 | | |
| Total | 47 | | | |

S = 183.030
R-Sq = 94.77%
R-Sq(adj) = 91.81%

TABLE 1B
Mean differences for payable yield (lower diagonal) and P values
(upper diagonal) for Bonferroni pairwise comparisons

| | Golden Hills | Kerman | S-32 | S-43 |
|---|---|---|---|---|
| Golden Hills | — | 0.0003 | 0 | 0.0289 |
| Kerman | −350 | — | 0.0008 | 0.6787 |
| S-32 | −677 | −327 | — | — |
| S-43 (Gumdrop) | −228 | 122 | 449 | — |

Color of leaves and nuts: For leaves, color evaluations were done on at least 3 leaves, each new and mature, collected at random from a ‘Gumdrop’ tree. Color descriptions are based on the color standards presented in Royal Horticultural Society; R.H.S. colour chart v. 3 QC495 R8 V.1-4. Mature leaves, top surface=139B, bottom surface=139B, new leaves, top surface=138B, 139C, bottom surface=139C, leaf midrib=143C. For ‘Gumdrop’ nuts, evaluations were done on 3 or more nuts, using the R.H.S. colour chart v. 3 for color standards, as described above. Husks as described are the exocarp outside of the shells, and kernel values are taken after shell removal. Husk tip=149D, 150D with some 145D, body=149D, 150D with some 145D, stem end=149D, 150D with some 145D, kernel face=80B, 80C, back =143D.

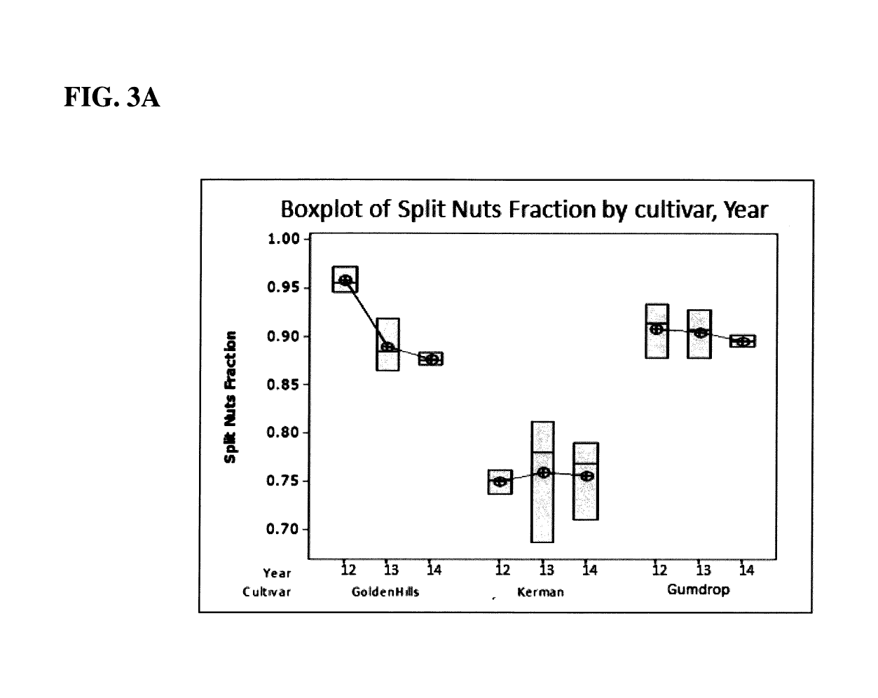

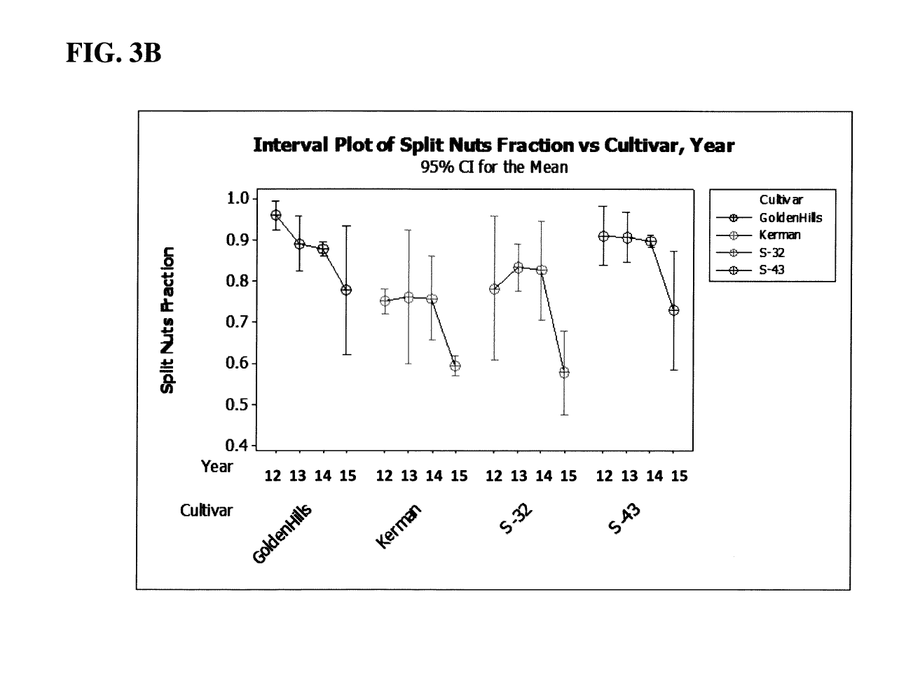

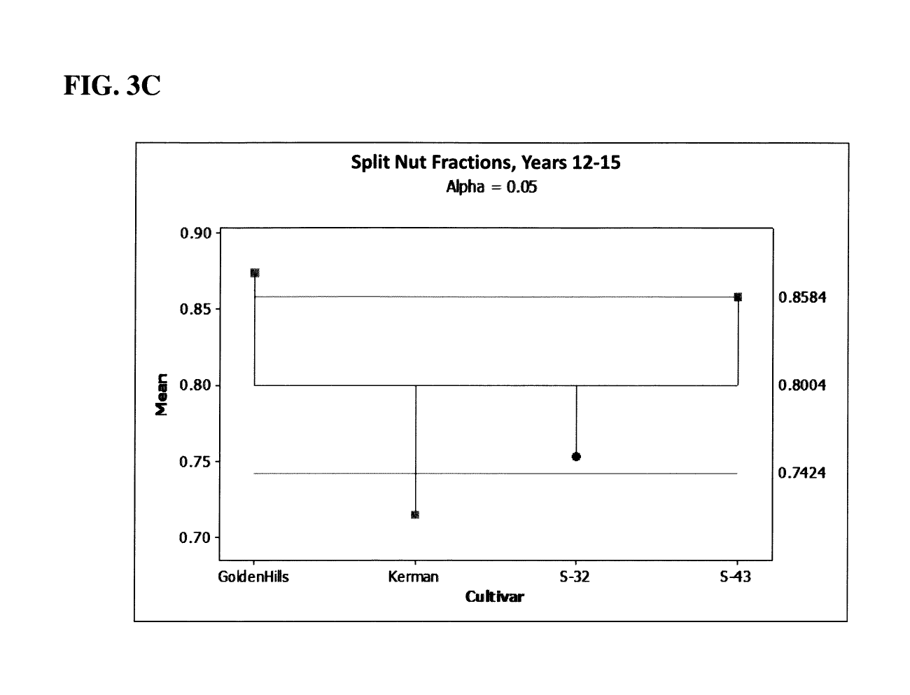

Nut Characteristics, Split nuts/Non-split nuts: Based on data from Years 12-14, ‘Gumdrop’ had a very high split percentage (90.2%), similar to ‘Golden Hills’ (90.7%) and significantly better (Bonferroni, Tukey tests) than ‘Kerman’ (75.6%) (FIG. 3A). Cultivars that split well are desired by growers since they receive substantially higher payment for naturally split nuts. Non-splits are usually processed for kernels to be used in confections.

Based on data from Years 12-15, ‘Gumdrop’ had a very high split percentage (85.8%), similar to ‘Golden Hills’ (87.4%) and significantly better (Bonferroni test) than ‘Kerman’ (71.4%). An analysis and statistics summary is provided in Table 2A and Table 2B. These differences were especially notable during Year 15 (FIG. 3B), when ‘Kerman’ showed a very high non-split percentage, as well as low yields. Split percentage for ‘Gumdrop’ was comparable to ‘Golden Hills’ (FIG. 3C), which is the “gold standard” for splits. Split percentages were generally low in Year 15 for all varieties analyzed due to insufficient chilling, producing more blanks, etc.

TABLE 2A
Analysis of Variance for Split Nut Fractions

| Source | DF | MS | F | P |
|---|---|---|---|---|
| Cultivar | 3 | 0.073604 | 44.24 | 0.000 |
| Year | 3 | 0.094055 | 56.54 | 0.000 |
| Cultivar * Year | 9 | 0.003199 | 1.92 | 0.087 |
| Block | 2 | 0.000353 | 0.21 | 0.810 |
| Error | 30 | 0.001664 | | |
| Total | 47 | | | |

S = 0.0407876
R-Sq = 91.43%
R-Sq(adj) = 86.57%

TABLE 2B
Mean differences for fraction of split nuts (lower diagonal) and P values
(upper diagonal) for Bonferroni pairwise comparisons

| | Golden Hills | Kerman | S-32 | S-43 |
|---|---|---|---|---|
| Golden Hills | — | 0.0000 | 0.0000 | 1.0000 |
| Kerman | −0.16 | — | 0.1575 | 1.0000 |
| S-32 | −0.121 | 0.039 | — | 0.0000 |
| S-43 (Gumdrop) | −0.012 | 0.144 | 0.105 | — |

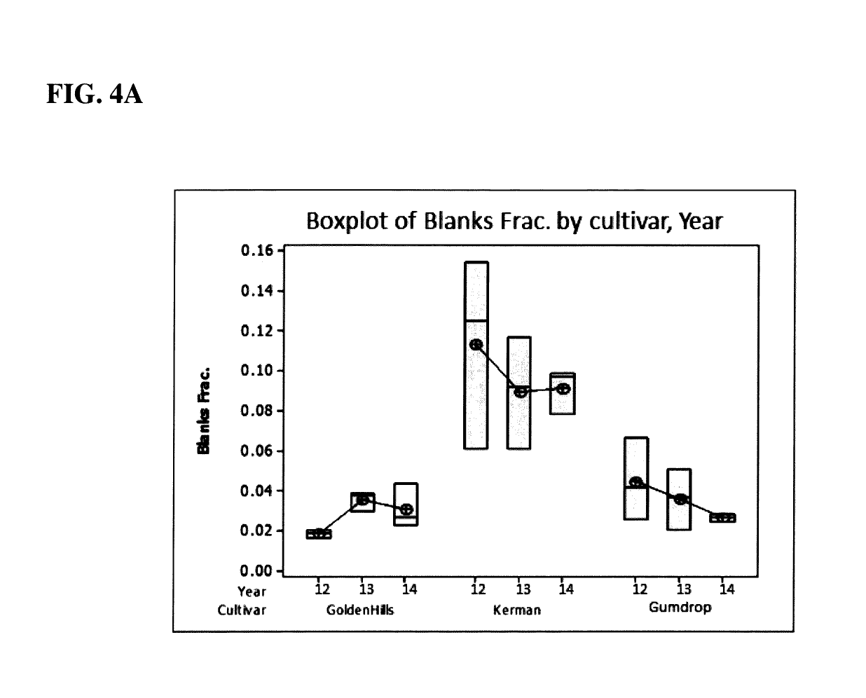

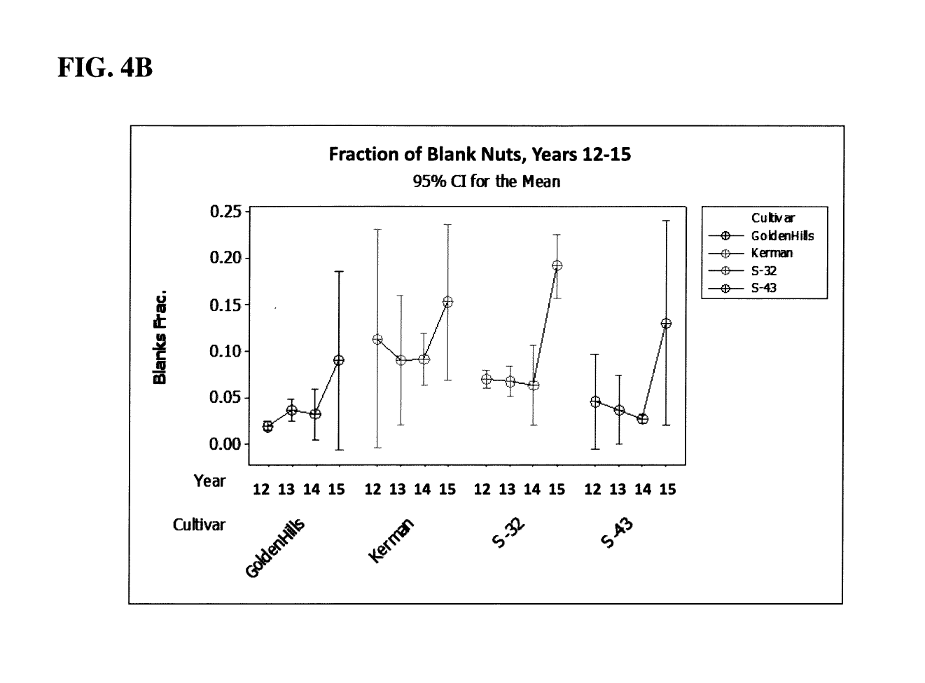

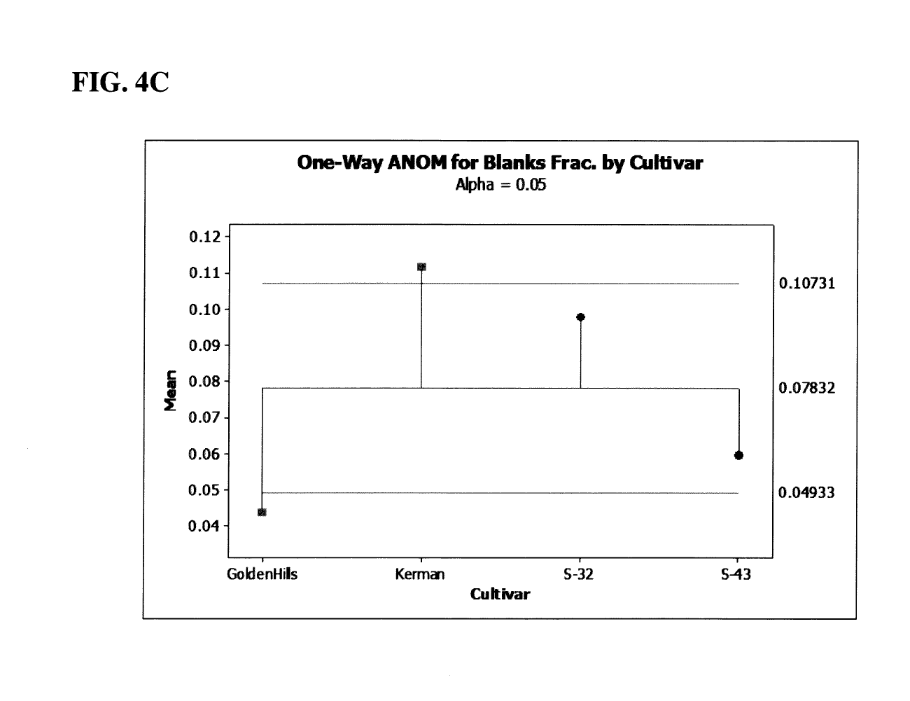

Nut Characteristics, Blank nuts: Based on data from Years 12-14, ‘Gumdrop’ had a low level of blank nuts (fully formed shell but no kernel). Growers are not paid for blanks. Some genotypes have naturally high levels of blanks and give the impression of high yield prior to harvest. ‘Gumdrop’ was similar (3.6%) to ‘Golden Hills’ (2.9%) but significantly less (Bonferroni, Tukey tests) than ‘Kerman’ (9.8%) (FIG. 4A).

Year 15 was an exceptional year and all of the tested cultivars had unusually high blank percentages (‘Kerman’=15.3%, ‘Golden Hills’=8.9%, ‘S-43’=13.0%). ‘Gumdrop’ had a low level of blank nuts from Years 12-14 (3.6%), similar to ‘Golden Hills’ (2.9%) but significantly less (Bonferroni) than ‘Kerman’ (9.8%). An analysis and statistics summary is provided in Table 3A and Table 3B. ‘Kerman’ has a reputation for producing blanks, and over the 4 years of data collection (Years 12-15), averaged 11.2%. Across all 4 years of the trial (Years 12-15), ‘Gumdrop’ had significantly fewer blanks than ‘Kerman’ (5.97% vs. 11.2%), and similar values to ‘Golden Hills’ (4.4%) (FIG. 4B and FIG. 4C).

TABLE 3A
Analysis of Variance for Blanks Fraction

| Source | DF | MS | F | P |
|---|---|---|---|---|
| Cultivar | 3 | 0.0121874 | 23.83 | 0.000 |
| Year | 3 | 0.0210644 | 41.19 | 0.000 |
| Cultivar * Year | 9 | 0.0009813 | 1.92 | 0.087 |
| Block | 2 | 0.0014740 | 2.88 | 0.072 |
| Error | 30 | 0.0005113 | | |
| Total | 47 | | | |

S = 0.0226130
R-Sq = 87.91%
R-Sq(adj) = 81.06%

TABLE 3B
Mean differences for blank fractions (lower diagonal) and P values
(upper diagonal) for Bonferroni pairwise comparisons

| | Golden Hills | Kerman | S-32 | S-43 |
|---|---|---|---|---|
| Golden Hills | — | 0.0000 | 0.0000 | 0.5749 |
| Kerman | 0.068 | — | 0.8780 | 0.0000 |
| S-32 | 0.054 | −0.014 | — | |
| S-43 (Gumdrop) | 0.016 | −0.052 | −0.038 | — |

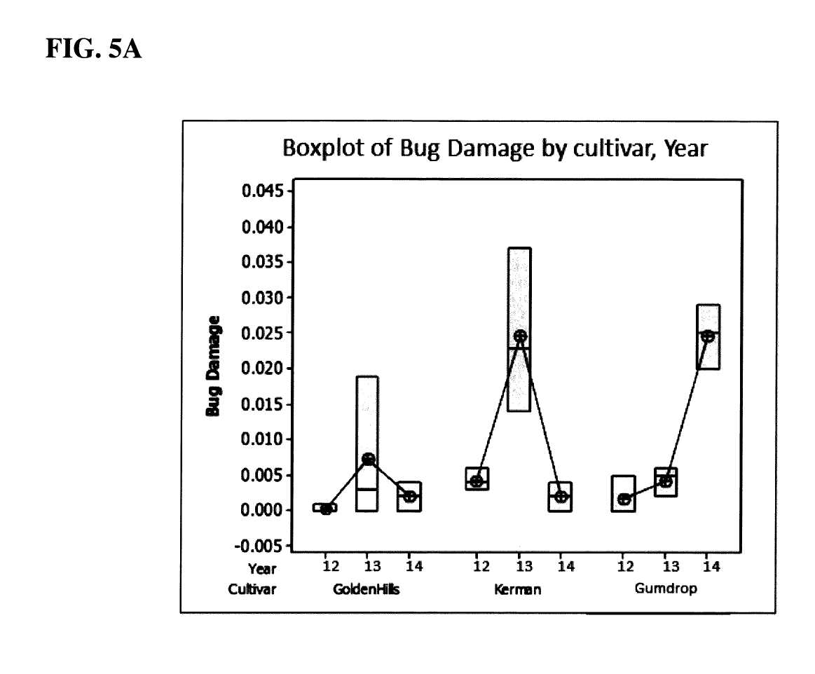

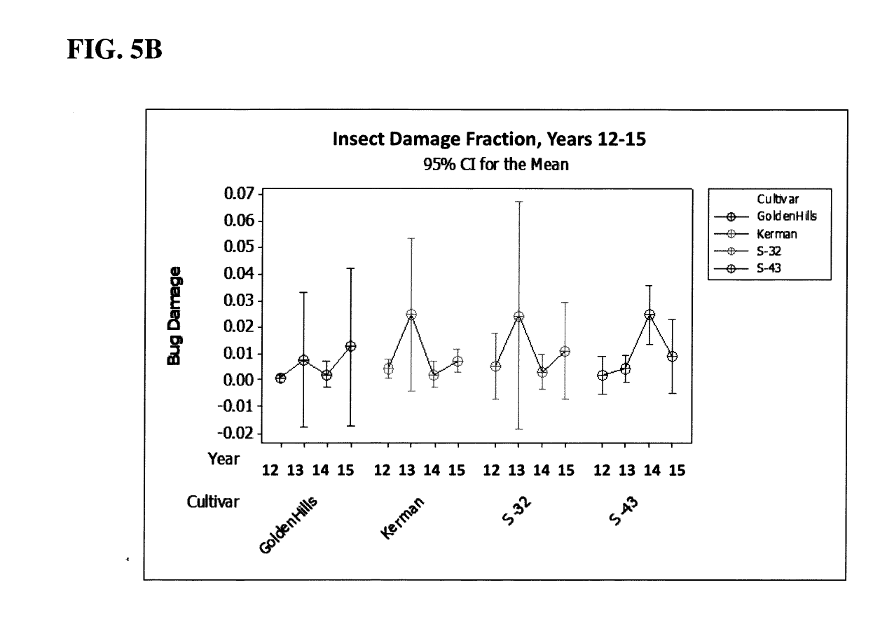

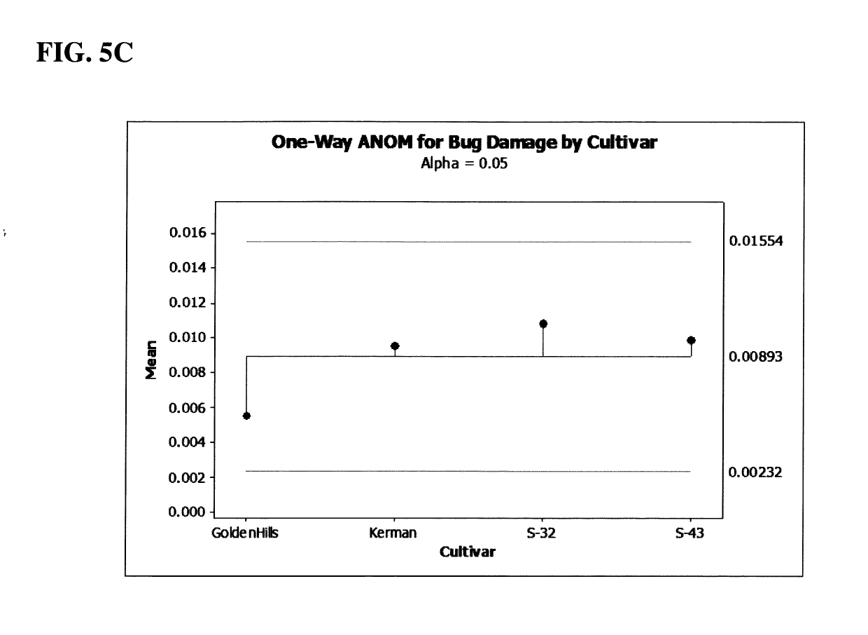

Nut Characteristics, Insect damaged nuts: Insect damaged nuts are considered a defect. Bug damage was generally very low in the plot analyzed (0.5%), except for Year 13, when ‘Golden Hills’ and ‘Kerman’ had significant damage (>2.0%). Overall, bug damage for ‘Gumdrop’ was not significantly different among cultivars (GLM ANOVA), but year by cultivar (year*cultivar) interactions were significantly different based on the analysis for Years 12-14 (FIG. 5A). ‘Gumdrop’ had low bug damage in Year 13, but high levels in Year 14. This discrepancy may be due to differences in harvest date among the cultivars and/or different types of insect activity during the pre-harvest period.

The combined data for Years 12-15 were also analyzed. Insect damage remained very low in the plot analyzed (0.9%), even through Year 15, again with the exception of Year 13 as described above (FIG. 5B and FIG. 5C). ‘Golden Hills’ also had relatively high levels of insect damage in Year 15. Overall, bug damage was not significantly different among cultivars, but cultivar by year (cultivar*year) interactions were significantly different. An analysis summary is provided in Table 4. ‘Gumdrop’ had low bug damage in Year 13, but somewhat higher levels in both Year 14 and Year 15. The harvest of ‘Gumdrop’ was delayed again in Year 15, as was also the case in past years, because no processor was available at the early time of ‘Gumdrop’ maturity to process the nuts. A timely harvest is likely to reduce insect damage and shell staining. Ensuring a timely harvest is likely to reduce the degree of insect damage and shell staining. The nuts in this trial (and the whole orchard) were not treated for navel orangeworm due to poor yields of ‘Kerman’ in the oil-treated portion of the block in Year 15. The trial area was not oiled in its entirety.

TABLE 4
Analysis of Variance for Bug Damage

| Source | DF | MS | F | P |
|---|---|---|---|---|
| Cultivar | 3 | 0.0000661 | 1.29 | 0.294 |
| Year | 3 | 0.0003113 | 6.09 | 0.002 |
| Cultivar * Year | 9 | 0.0002306 | 4.51 | 0.001 |
| Block | 2 | 0.0000775 | 1.52 | 0.236 |
| Error | 30 | 0.0000511 | | |
| Total | 47 | | | |

S = 0.00714777
R-Sq = 68.69%
R-Sq(adj) = 50.95%

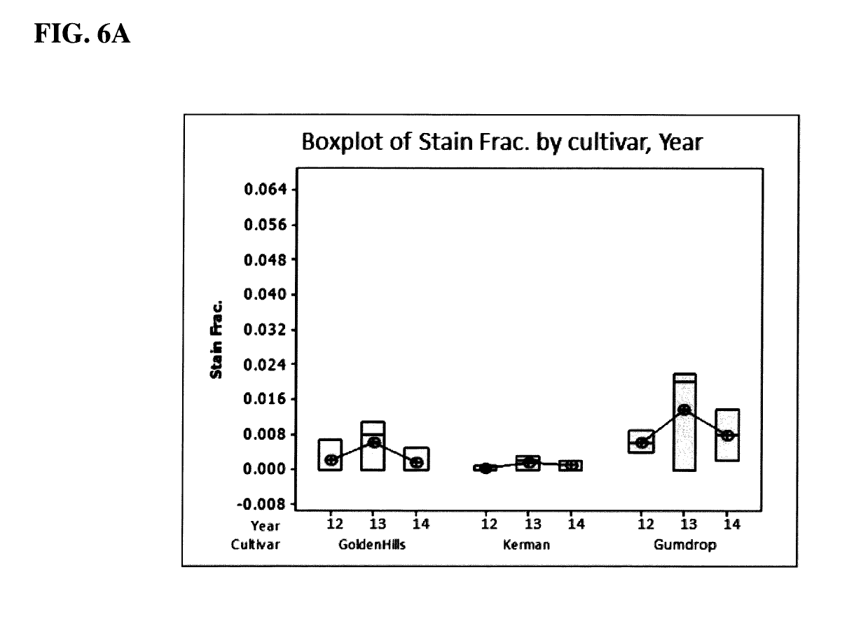

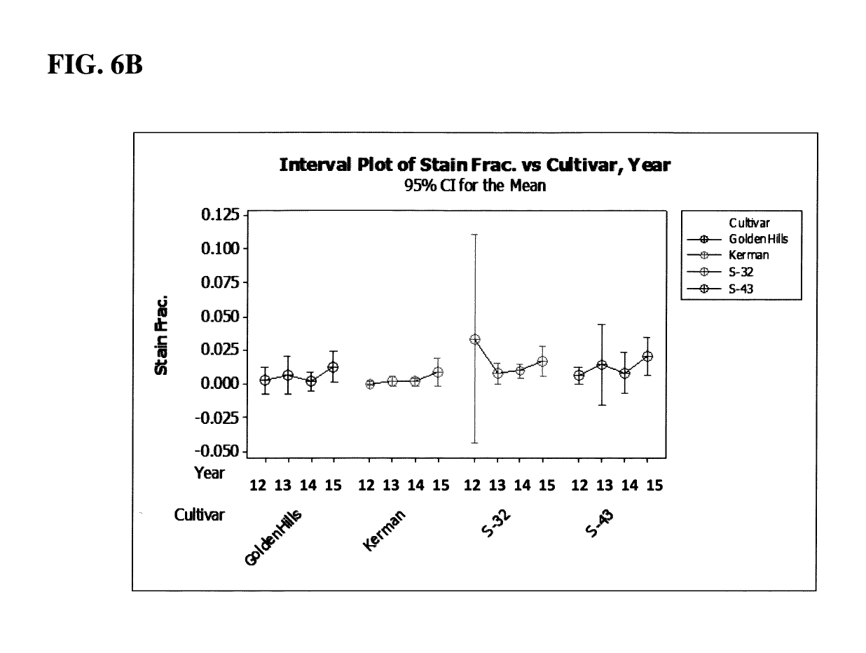

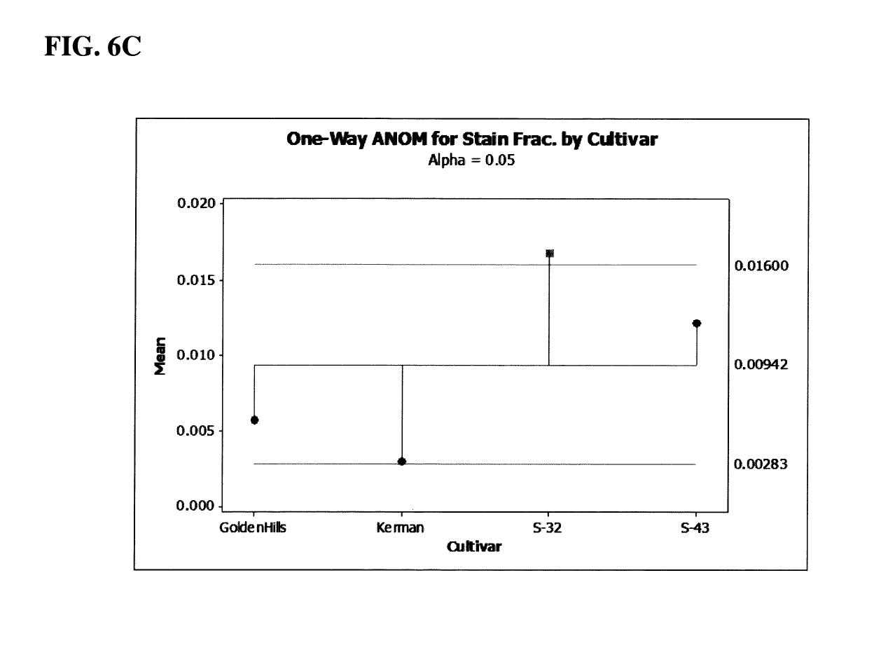

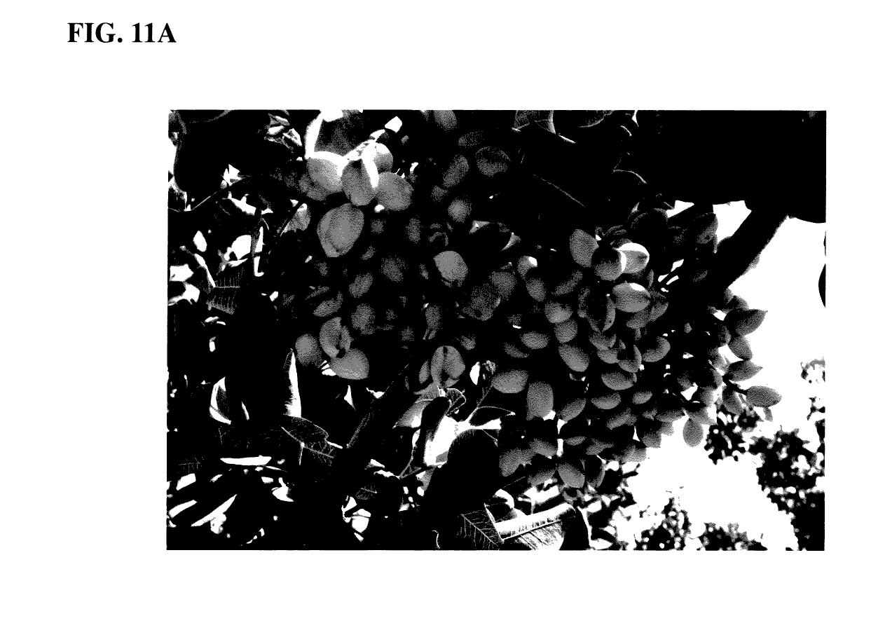

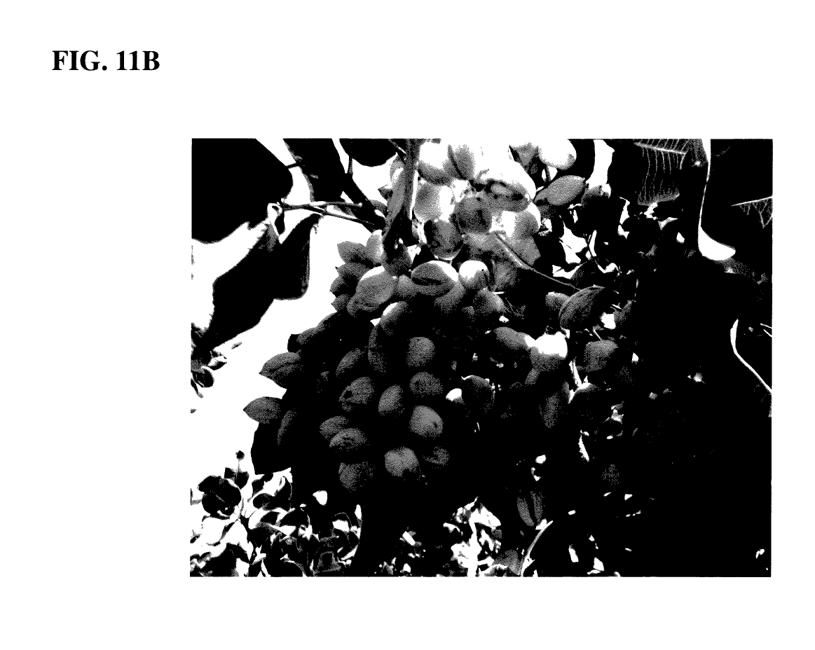

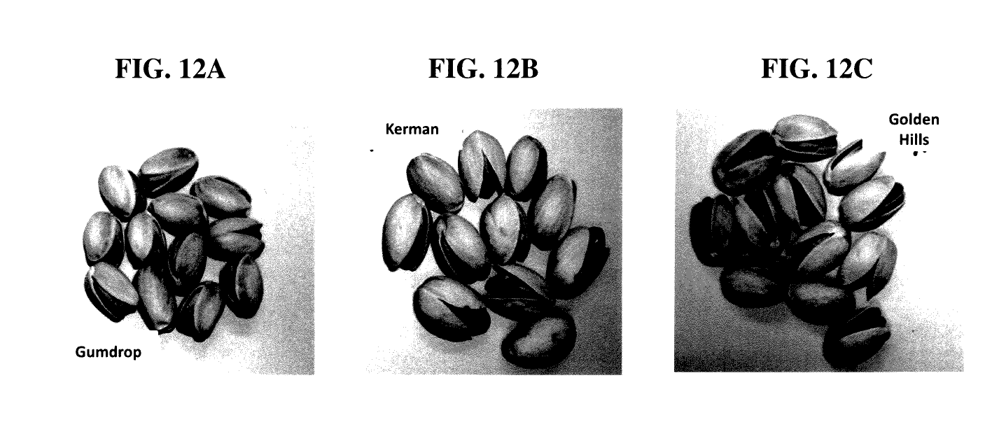

Nut Characteristics, Shell staining: Shell staining is an important characteristic, impacting consumer acceptance. Shell staining is the reason, along with bug damage, that Iranian pistachio cultivars were stained red—to cover the defect. For Years 12-14, shell staining was minimal for all of the cultivars, except for ‘S-32’ in the first harvest year (Year 12). ‘Gumdrop’ had somewhat higher (non-significant by Bonferroni, Tukey tests) stain fraction (0.9%) than ‘Kerman’ (0.1%) or ‘Golden Hills’ (0.3%) (FIG. 6A and FIG. 12A). For Years 12-15, ‘Gumdrop’ had somewhat higher (non-significant by Bonferroni) stain fraction (1.22%) than ‘Kerman’ (0.30%) or ‘Golden Hills’ (0.575%) (FIG. 6B and FIG. 6C). An analysis and statistics summary is provided in Table 5A and Table 5B. Views of the ‘Gumdrop’ nut clusters at maturity are presented in FIG. 11A. FIG. 11B illustrates an additional view of ‘Gumdrop’ nut clusters.

TABLE 5A
Analysis of Variance for Stain Fraction

| Source | DF | MS | F | P |
|---|---|---|---|---|
| Cultivar | 3 | 0.0004638 | 5.86 | 0.003 |
| Year | 3 | 0.0002033 | 2.57 | 0.073 |
| Cultivar * Year | 9 | 0.0001503 | 1.90 | 0.091 |
| Block | 2 | 0.0001626 | 2.05 | 0.146 |
| Error | 30 | 0.0000792 | | |
| Total | 47 | | | |

S = 0.00889975
R-Sq = 60.76%
R-Sq(adj) = 38.53%

TABLE 5B
Mean differences for stain fractions (lower diagonal) and P values
(upper diagonal) for Bonferroni pairwise comparisons

| | Golden Hills | Kerman | S-32 | S-43 |
|---|---|---|---|---|
| Golden Hills | — | 1.0000 | 0.0302 | 0.5254 |
| Kerman | −0.003 | — | 0.0041 | 0.1030 |
| S-32 | 0.011 | 0.014 | — | |
| S-43 (Gumdrop) | 0.006 | 0.009 | −0.004 | — |

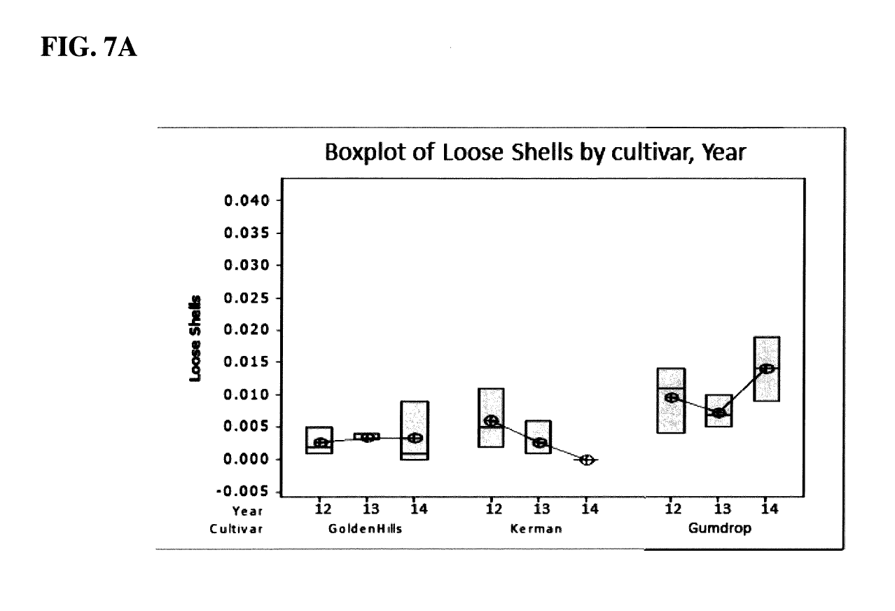

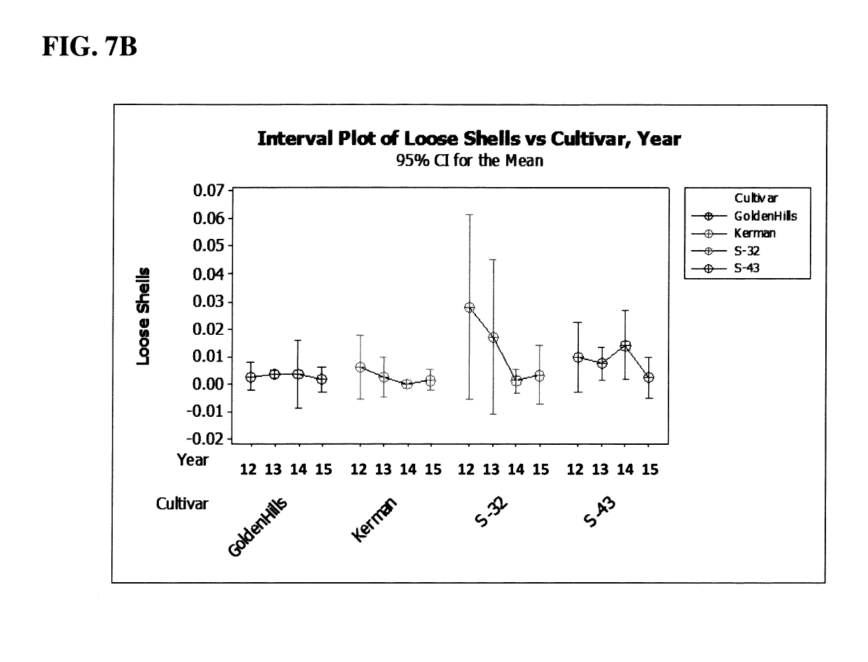

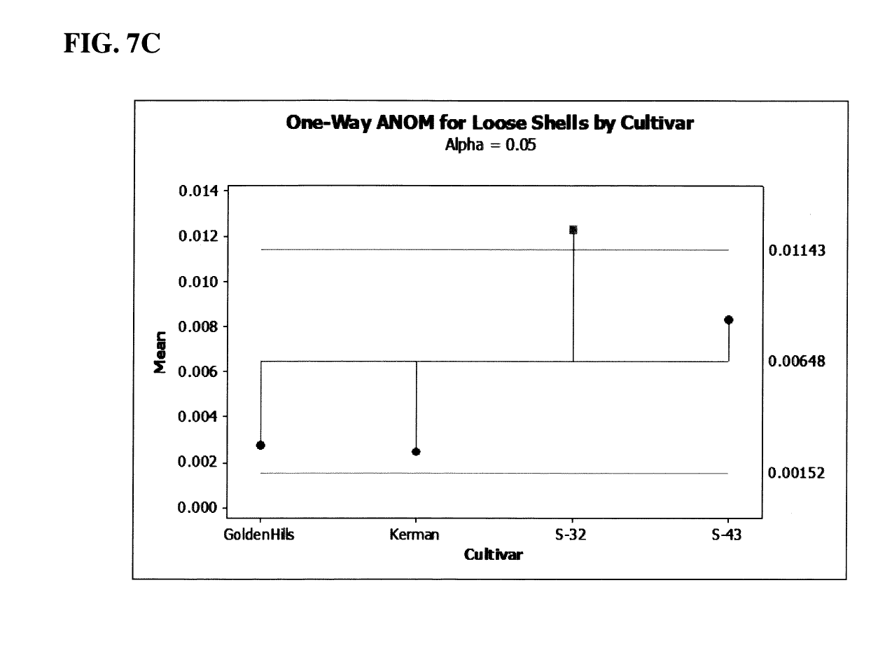

Nut Characteristics, loose shells: The loose shell evaluation measures the tendency of the husked nuts to lose their shells during or after processing, resulting in loose kernels and shell pieces in the commercial in-shell product stream. Loose shells are a function of shell hinge strength. If hinge strength is exceptionally high (no loose shells), consumers will have difficulty extracting the nuts from the shells. For this reason, hinge strength should be at an intermediate level, rather than extremely strong or weak. For Years 12-14, ‘Gumdrop’ had higher, but non-significantly different (Bonferroni, Tukey tests), levels (1.0%) of loose shells than ‘Kerman’ (0.3%) or ‘Golden Hills’ (0.3%) (FIG. 7A). For Years 12-15, ‘Gumdrop’ had higher, but non-significantly different (Bonferroni test), levels (0.84%) of loose shells than ‘Kerman’ (0.25%) or ‘Golden Hills’ (0.28%) (FIG. 7B and FIG. 7C). An analysis and statistics summary is provided in Table 6A and Table 6B. The appearance of husked and dried nuts of ‘Gumdrop’, ‘Kerman’, and ‘Golden Hills’ are presented in FIG. 12A, FIG. 12B, and FIG. 12C, respectively.

TABLE 6A
Analysis of Variance for Loose Shells

| Source | DF | MS | F | P |
|---|---|---|---|---|
| Cultivar | 3 | 0.0002694 | 9.11 | 0.000 |
| Year | 3 | 0.0001984 | 6.71 | 0.001 |
| Cultivar * Year | 9 | 0.0001239 | 4.19 | 0.001 |
| Block | 2 | 0.0000204 | 0.69 | 0.510 |
| Error | 30 | 0.0000296 | | |
| Total | 47 | | | |

S = 0.00543707
R-Sq = 74.27%
R-Sq(adj) = 59.69%

TABLE 6B
Mean differences for loose shell fractions (lower diagonal) and P values
(upper diagonal) for Bonferroni pairwise comparisons

| | Golden Hills | Kerman | S-32 | S-43 |
|---|---|---|---|---|
| Golden Hills | — | 1.0000 | 0.0010 | 0.1013 |
| Kerman | −0.0003 | — | 0.0007 | 0.0762 |
| S-32 | 0.0096 | 0.0098 | — | 0.5184 |
| S-43 (Gumdrop) | 0.0056 | 0.0059 | −0.0040 | — |

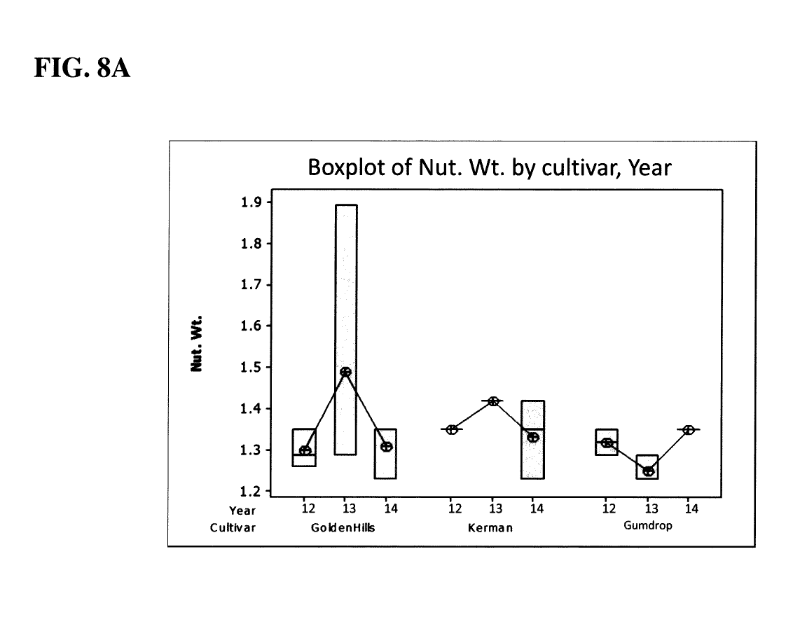

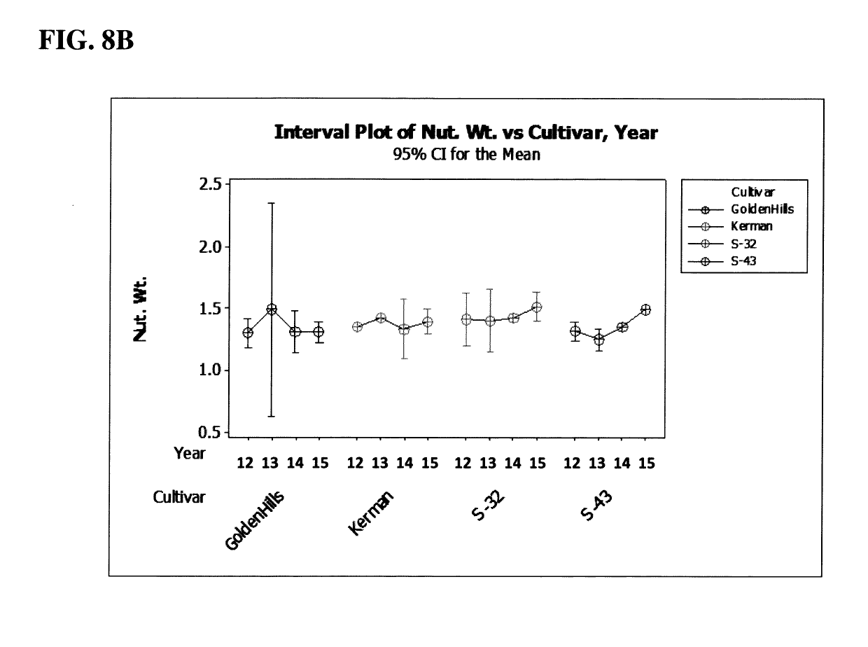

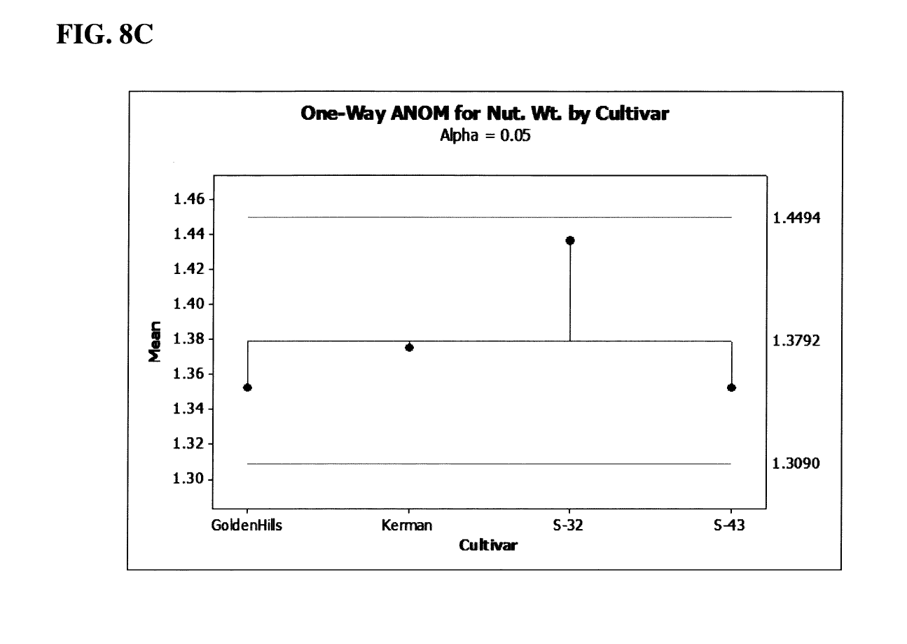

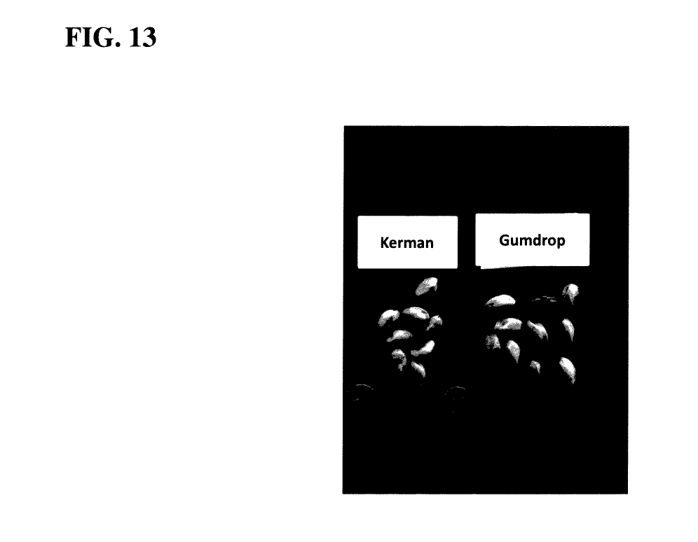

Nut Characteristics, nut weight (including shells): Nut weight, nut length, and nut width are correlated characters, so only nut weight as a measure of nut size is presented in the analysis for Years 12-14. Nut size was generally similar for all of the cultivars and years, except for ‘Golden Hills’ in harvest year 2 (Year 13). ‘Gumdrop’ had a mean nut weight of 1.31 g, and both ‘Kerman’ and ‘Golden Hills’ had a mean nut weight of 1.37 g (FIG. 8A). Cultivar and Year effects were not significantly different (ANOVA, Bonferroni, Tukey tests). Nut quality for ‘Gumdrop’ is similar to other cultivars, with the exception that the husks tend to be ‘gummy’ or sticky. However, processers have not indicated that this is a problem after processing, and the processed nuts for ‘Gumdrop’ are similar in appearance to ‘Kerman’ or ‘Golden Hills’. Views of isolated nut kernels from ‘Gumdrop’ and ‘Kerman’ are presented in FIG. 13.

The combined data for Years 12-15 were also analyzed. ‘Gumdrop’ had a mean nut weight of 1.35 g, ‘Kerman’ had a mean nut weight of 1.38 g, and ‘Golden Hills’ had a mean nut weight of 1.35 g (FIG. 8B and FIG. 8C). Cultivar and Year effects were not significantly different (by ANOVA, ANOMA, and Bonferroni paired comparisons). An analysis and statistics summary is provided in Table 7A and Table 7B.

TABLE 7A
Analysis of Variance for Nut Weight

| Source | DF | MS | F | P |
|---|---|---|---|---|
| Cultivar | 3 | 0.01898 | 1.85 | 0.160 |
| Year | 3 | 0.01748 | 1.70 | 0.188 |
| Cultivar * Year | 9 | 0.01729 | 1.68 | 0.137 |
| Block | 2 | 0.00661 | 0.64 | 0.533 |
| Error | 30 | 0.01028 | | |
| Total | 47 | | | |

TABLE 7B
Mean differences for nut weight in grams (lower diagonal) and P values
(upper diagonal) for Bonferroni pairwise comparisons

| | Golden Hills | Kerman | S-32 | S-43 |
|---|---|---|---|---|
| Golden Hills | — | 1.0000 | 0.3056 | 1.0000 |
| Kerman | 0.0225 | — | 0.8802 | 1.0000 |
| S-32 | 0.0842 | 0.0617 | — | 0.5184 |
| S-43 (Gumdrop) | 0.0000 | −0.0225 | −0.0842 | — |

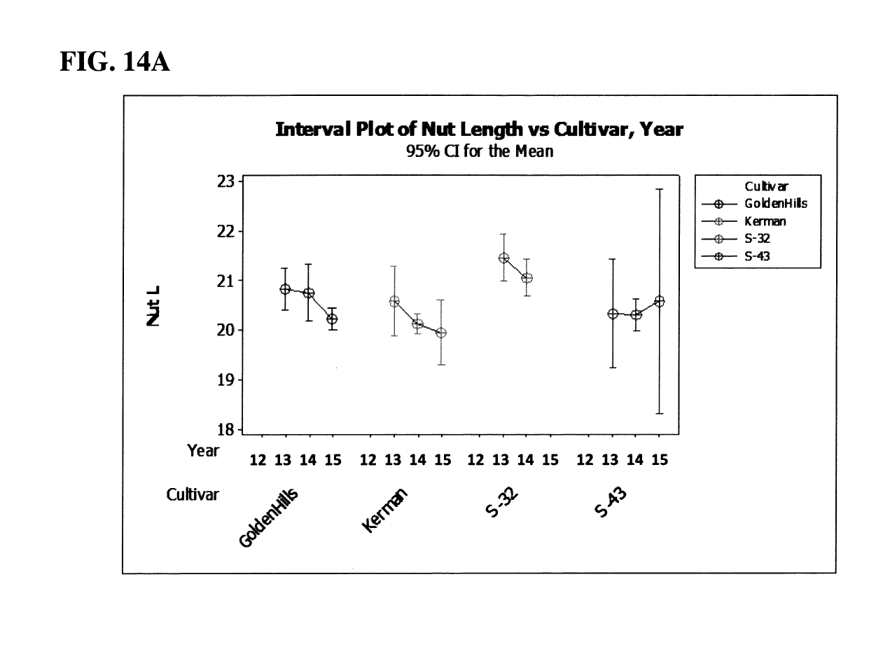

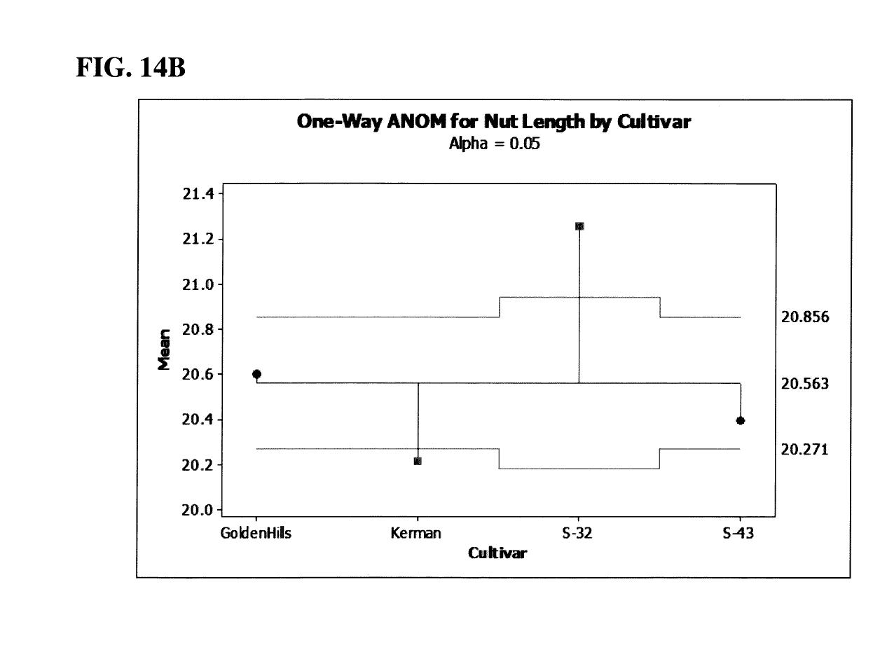

Nut Characteristics: nut length and nut width: Two parameters were measured from Year 13-Year 15: nut length and nut width (measured parallel to the split). Nut length values for ‘Gumdrop’ (‘S-43’), ‘Golden Hills’, and ‘Kerman’ were not significantly different (20.4 mm, 20.6 mm, and 20.22 mm, respectively) (FIG. 14A and FIG. 14B). ‘Gumdrop’ was intermediate in terms of nut length between ‘Kerman’ and ‘Golden Hills’, having a nut length difference of ˜0.2 mm from each of them. An analysis and statistics summary is provided in Table 8A and Table 8B.

TABLE 8A
Analysis of Variance for Nut Length

| Source | DF | Adj MS | F | P |
|---|---|---|---|---|
| Cultivar | 3 | 1.1294 | 8.20 | 0.001 |
| Year | 2 | 0.3323 | 2.41 | 0.110 |
| Block | 2 | 0.1232 | 0.89 | 0.422 |
| Error | 25 | 3.4452 | 0.1378 | |
| Total | 32 | | | |

S = 0.371226
R-Sq = 59.97%
R-Sq(adj) = 48.76%

TABLE 8B
Mean differences for nut length (lower diagonal) and P values
(upper diagonal) for Bonferroni pairwise comparisons

| | Golden Hills | Kerman | S-32 | S-43 |
|---|---|---|---|---|
| Golden Hills | — | 0.2191 | 0.0498 | 1.0000 |
| Kerman | −0.387 | — | 0.0004 | 1.0000 |
| S-32 | 0.579 | 0.966 | — | 0.004 |
| S-43 (Gumdrop) | 0.206 | 0.181 | −0.785 | — |

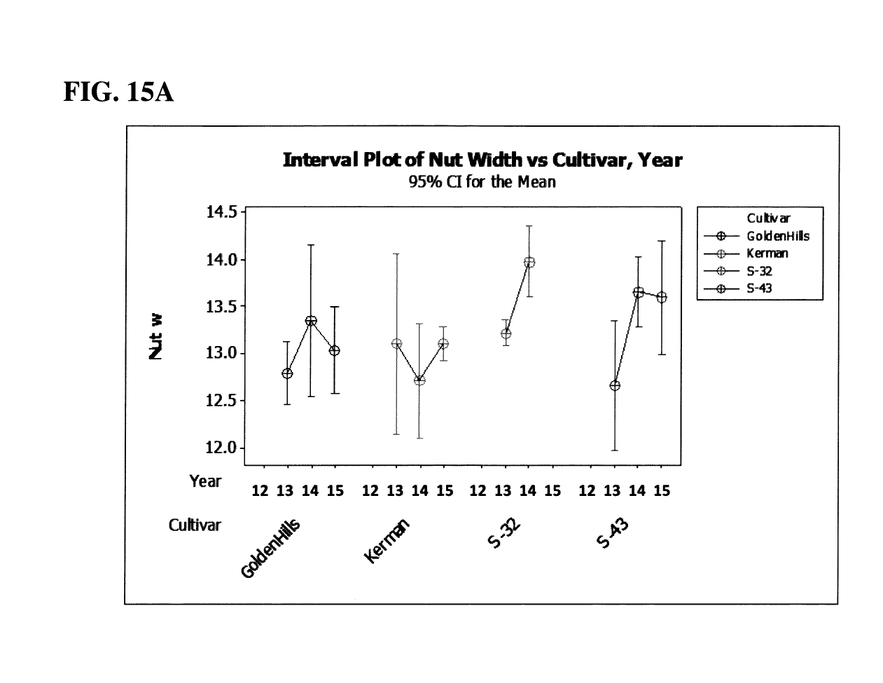

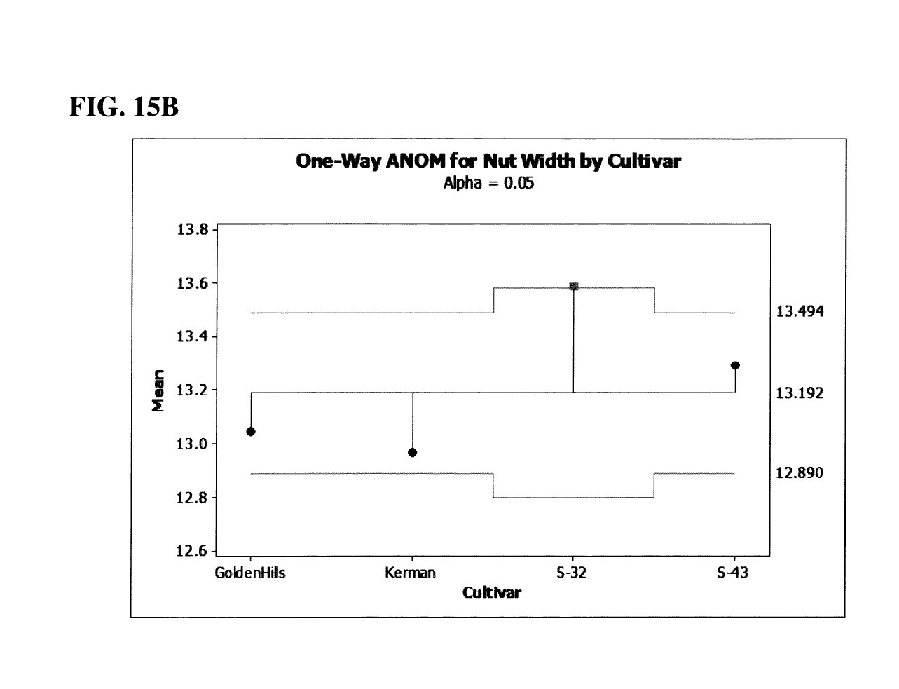

In terms of nut width, no significant differences in nut width were observed between ‘Gumdrop’ (‘S-43’), ‘Golden Hills’, and ‘Kerman’ (13.3 mm, 13.1 mm, and 13.0 mm, respectively), with ‘Gumdrop’ having the greatest width (FIG. 15A and FIG. 15B). However, ‘S-32’ was significantly different than the other cultivars for both length and width, having a larger nut than the other cultivars. An analysis and statistics summary is provided in Table 9A and Table 9B.

TABLE 9A
Analysis of Variance for Nut Width

| Source | DF | Adj MS | F | P |
|---|---|---|---|---|
| Cultivar | 3 | 0.6373 | 5.31 | 0.006 |
| Year | 2 | 0.8152 | 6.80 | 0.004 |
| Block | 2 | 0.0057 | 0.05 | 0.954 |
| Error | 25 | 0.1199 | | |
| Total | 32 | | | |

S = 0.346327
R-Sq = 52.64%
R-Sq(adj) = 39.38%

TABLE 9B
Mean differences for nut width (lower diagonal) and P values
(upper diagonal) for Bonferroni pairwise comparisons

| | Golden Hills | Kerman | S-32 | S-43 |
|---|---|---|---|---|
| Golden Hills | — | 1.0000 | 0.0204 | 0.8498 |
| Kerman | −0.081 | — | 0.0070 | 0.3290 |
| S-32 | 0.610 | 0.691 | — | 0.3969 |
| S-43 (Gumdrop) | 0.248 | 0.329 | −0.362 | — |

What is claimed is:

1. A new and distinct variety of pistachio tree designated ‘Gumdrop’ as shown and described herein.
